(19) United States
(12) Patent Application Publication
(10) Pub. No.: US 2012/0177751 A1
(43) Pub. Date: Jul. 12, 2012

(54) Pediculosis Treatment and Prophylaxis

(75) Inventors: Wendy Sue Langley, Albany, OR (US); Nikki Lynn Frum, Albany, OR (US); Vernon Wade Smith, Albany, OR (US); Larry Mark Burris, Albany, OR (US); Steven Dale Smith, Corvallis, OR (US)

(73) Assignee: TEC LABORATORIES, INC., Albany, OR (US)

(21) Appl. No.: 13/344,937

(22) Filed: Jan. 6, 2012

Related U.S. Application Data
(60) Provisional application No. 61/430,397, filed on Jan. 6, 2011.

(51) Int. Cl. A61K 33/14; A61P 33/14; A01P 15/00; A01N 59/08

(52) U.S. Cl. 424/680

(57) ABSTRACT

Disclosed is a non-toxic composition and methods for controlling and preventing lice infestations. The composition of the invention controls louse populations with daily use. The composition of the invention may be used on the hair, body, and on inanimate objects. The viscous liquid composition of the invention may be applied as a shampoo, body cleanser, or detergent.

### CROSS-REFERENCE TO RELATED APPLICATIONS: This application claims the benefit of provisional patent application number U.S. 61/430,397 filed Jan. 6, 2011 by the present inventors.

### FEDERALLY SPONSORED RESEARCH OR DEVELOPMENT: None.

### SEQUENCE LISTING: None.

### FIELD OF THE INVENTION

The present invention is in the technical field of lice control. More particularly, the present invention is in the technical field of non-toxic pediculosis treatment and prophylactic compositions and methods of use.

### BACKGROUND OF THE INVENTION

Lice can affect people of any income or social level. Experts agree it is especially difficult, if not impossible, to prevent head lice in particular from spreading among children because they commonly share hats, combs, and other items. Experts offer limited advice for prevention of head lice, and recommend frequent examination of the scalps of school-age children to help discover and treat lice before they spread to the rest of the family and to avoid prolonged contact with those infested.

Pubic lice are spread primarily among people who have had many sex partners. The advice given by experts for prevention of pubic lice is to reduce the number of sex partners to reduce the risk of getting lice.

Body lice are spread through human to human contact and from contact with contaminated clothing. Experts suggest that body lice may be prevented by bathing regularly, changing clothes daily, and washing clothes in hot water.

Experts agree that the best approach to lice prevention is to take steps to get rid of the lice so there doesn't become more to deal with.

Several products that claim to prevent and/or control head lice are available over the counter; the most widely used products in this category include products that contain toxic pesticides. Most products in this category are applied as shampoos or conditioners and must remain on the head for lengthy periods of time. Compositions for lice control have been introduced that are claimed to repel lice and/or aid in nit removal and have no pediculicidal effect.

### SUMMARY OF THE INVENTION

The present invention discloses non-toxic conditioning shampoo compositions having pediculicidal and prophylactic properties and methods of use to control and prevent head, body and pubic lice.

Further, the present invention is used every day to replace a person's regular shampoo and conditioner and/or body soap and when applied directly to the wet head or body of an affected person and then rinsed off, effectively controls all forms of human lice. In addition to a pediculicidal effect, the present invention also has a benefit of leaving behind an environment that lice find undesirable and uninhabitable, which causes any lice that might find their way to the treated area by way of shared hats and combs to leave as soon as a suitable host is near, thereby preventing an infestation. The composition also makes the environment unsuitable for louse egg laying, further preventing an infestation. Further, the present invention is simply applied to the wet affected area and used like a regular daily shampoo or soap by washing, rinsing, and repeating to control and prevent all types of human lice, including head, body, and pubic lice, so there is no disruption of normal, daily routine.

### DETAILED DESCRIPTION OF THE INVENTION

Referring now to the invention in more detail, the viscous liquid composition of this invention comprises water, a salt or combination of salts present in an amount effective to kill lice, a natural or synthetic oil or combination of oils that condition hair and skin and penetrate the hair shaft and louse exoskeleton, a conditioning agent or combination of conditioning agents, a fragrance fixative or combination of fixatives, a surfactant or combination of surfactants to allow for foaming, cleansing, and solubilization of the oils, at least one plant essence, an optional chelating agent, and at least one optional perfume.

In more detail, the salt of the instant invention comprises at least one ionic salt in an amount ranging from about 0.0001 to about 50 weight percent. The oil or combination of oils may be selected from a variety of known and available pharmaceutically acceptable materials and present in an amount up to about 60%. The conditioning agent or combination of conditioning agents may be selected from a variety of known and available pharmaceutically acceptable materials and present in an amount up to about 50%. The fragrance fixative of the instant invention may be selected from a variety of known and available pharmaceutically acceptable materials and present in an amount ranging from about 0.1 to about 20 weight percent. The surfactants or combination of surfactants of the instant invention may be selected from a variety of known and available pharmaceutically acceptable materials and present in the amount ranging from about 0.1 to about 75 weight percent. The plant essence of the instant invention may be selected from a variety of at least one plant essence, including but not limited to extracts, essential oils, and tinctures, derived from a plant or synthetic and present in an amount from about 0.01 to about 20 weight percent. The optional chelating agent may be selected from a variety of known and available pharmaceutically acceptable materials and present in an amount up to about 1 weight percent. The optional perfume or perfumes of the instant invention may be selected from a variety of known and available pharmaceutically acceptable natural or synthetic materials and present in an amount up to about 15 weight percent.

In further detail, a preferred amount of salt or combination of salts present in the composition of the instant invention may range from about 0.01 to about 10 weight percent. An even more preferred amount is 10 weight percent. Preferred salts of the instant invention are at least one salt selected from the group consisting of sodium chloride, potassium chloride, magnesium chloride, potassium iodide, sodium iodide, potassium iodide, ammonium chloride, zinc acetate, and calcium chloride. An especially preferred salt is sodium chloride. A preferred amount of natural or synthetic oil or combination of oils present in the combination of the instant invention may range from about 1 to about 25 weight percent. An even more preferred amount is 15 weight percent. Preferred oils of the composition of the instant invention are at least one natural or synthetic oil selected from the group consisting of coconut oil, palm kernel oil, palm oil, soybean oil, olive oil, mineral oil, sesame oil, peanut oil, fractionated coconut oil, canola oil, corn oil, and vegetable oil. An especially preferred oil is coconut oil.

A preferred amount of conditioning agent or combination of conditioning agents present in the composition of the instant invention may be up to about 25 weight percent. An even more preferred amount is 5 weight percent. Preferred conditioning agents of the composition of the instant invention are at least one selected from the group consisting of fractionated coconut oil, caprylic/capric triglycerides, propylene glycol, glycerin, silicones and silicone compounds, polymers and copolymers, proteins, and quaternary agents and quaternary compounds. An especially preferred conditioning agent is fractionated coconut oil.

Furthermore, a preferred amount of fragrance fixative or combination of fixatives present in the composition of the instant invention may range from about 1 to about 10 weight percent. An even more preferred amount is 5 weight percent. Preferred fixatives of the instant invention are at least one selected from the group consisting of benzyl alcohol, benzoin, benzyl benzoate, sandalwood, myrhh, vanilla, Balsam of Peru, musk, grape seed oil, castor oil, cedar wood, galbanum, labdanum, opoponax, and vetiver. An especially preferred fragrance fixative is benzyl alcohol.

A preferred amount of surfactant or combination of surfactants present in the composition of the instant invention may range from about 1 to about 50 weight percent. An even more preferred amount is 37 weight percent. Preferred surfactants of the instant invention are at least one surfactant selected from the group consisting of alcohols, alkanolamides, alkanolamines, sulfates, alkylbenzenes, olefins, acetates, oxides, amines, sulfonates, betaines, polymers, alkylphenols, fatty acids, poloxamers, phenols, glycols, imidazolines, isothionates, lanolin, lecithin, lignin, anhydrides, methyl esters, glycerides, stearates, glycols, phenols, quaternary surfactants, sarcosine, silicone, soaps, sodium, sorbitan, sugars, oils, sorbates, polysorbates, octoxynols, nonyl phenyl ethoxylates, sodium lauryl sarcosinate, lanolin alcohol, and their pharmaceutically acceptable derivatives, ethers, esters, sulfates, ethoxylates, oils, extracts, and/or salts. An especially preferred surfactant or combination of surfactants is a combination of cocomidopropyl betaine and 70% active sodium laureth sulfate.

A preferred amount of plant essence (which include extracts, essential oils, and tinctures) or combination of plant essences in the composition of the instant invention may range up to about 5 weight percent. An even more preferred amount is 3.3 weight percent. Preferred plant essences used in the composition of the instant invention may be selected from the group consisting of the essence of oregano, thyme, anise, lemongrass, tea tree (melaleuca), cinnamon bark, cinnamon leaf, eucalyptus, camphor, geranium, neem, ylang-ylang, azalea, lavender, garlic, birch tar, citronella, sabadilla, mezereum, staphysagria, ledum, sage, habanera, jojoba, walnut, peppermint, ginger, lemon, rosemary, clove, echinacea, geranium, rose, and chrysanthemum. An especially preferred plant essence is or combination of plant essences is a combination of tea tree oil, lavender oil, anise oil, and peppermint oil.

A preferred amount of optional chelating agent in the composition of the instant invention may range up to about 5 weight percent. An even more preferred amount is 0.2 weight percent. Preferred chelating agents used in the composition of the instant invention may be selected from the group consisting of ethylenediaminetetraacetate, diethylenetriaminepentaacetate, aminocarboxylate, and their associated salts. An especially preferred optional chelating agent is disodium ethylenediaminetetraacetate (disodium EDTA).

A preferred amount of optional fragrance or combination of fragrances in the composition of the instant invention may range up to about 0.5 weight percent. An even more preferred amount is 0.2 weight percent. Any fragrance may be used. An especially preferred optional fragrance is ylang ylang.

Several compositions have been formulated with combinations of the various ingredients listed above. A preferred formulation contains 13 weight percent cocamidopropyl betaine (Norman Fox & Co.), 15 weight percent edible coconut oil (WHC), 24 weight percent 70% active sodium laureth sulfate (Norfox SLES 70), 5 weight percent fractionated coconut oil (Norman Fox & Co.), 2.5 weight percent tea tree oil (Naturex), 0.3 weight percent lavender oil (Berje), 0.3% anise oil (Berje), 0.2% peppermint oil (RCB), 0.2% ylang ylang oil (Berje), 24.3 weight percent purified water, 5 weight percent benzyl alcohol (Univar), 0.2 weight percent disodium EDTA (Dow), and 10 weight percent sodium chloride (Morton). The ingredients are added to a mixing vessel in the order given. The mixer is started after the addition of the fractionated coconut oil and remains on at moderate speed while adding the rest of the ingredients and then until uniform and viscous.

Surprisingly, during the formulation development process, a stable formula was created using a very high oil concentration in water with a limited amount of surfactant, with absolutely no oil/water separation even at high temperature (48° C. for 30 days). Also, it is warned by a supplier of 70% active sodium laureth sulfate (SLES), Norman Fox & Co., that SLES should be added to water, and never the other way around. This formulation safely and successfully adds water to the SLES already in the mixing vessel and creates an elegant and stable final product. This invention also includes several methods of use. One method of use is as a shampoo to control a louse infestation on human hair and body parts. The composition of the instant invention is housed in any type of container providing application of a viscous liquid, such as a tube. The composition of the invention is applied to affected wet hair or body parts and worked into a lather then rinsed, during which time the louse exoskeleton is stripped of its protective waxy covering and the louse becomes vulnerable to the environment. This process is then repeated, during which time the exposed lice are exterminated or permanently damaged. After rinsing, the hair and body is left in a state that lice find undesirable, uninhabitable, and unfit for egg laying. It is recommended, but not required, to let the composition remain on the affected hair or body parts for about three minutes before rinsing during the second application. The shampoo should be used daily for 7-14 days to control any newly hatched lice.

Another method of use is as a prophylactic to prevent louse populations from developing on the hair or body of an individual. The composition of the instant invention is housed in any type of container for dispensing viscous liquids, such as a tube. The composition of the invention is applied to wet hair or body parts and worked into a lather and rinsed off, during which time the louse's exoskeleton is stripped of its protective waxy covering and the louse becomes vulnerable to the environment. This wash then rinse process is repeated, during which time the louse is exterminated or permanently damaged. After rinsing, the hair and body are left in a state that lice find undesirable, uninhabitable, and unfit for egg laying.

Further, another method of use is to add a small amount of the composition of the instant invention to the laundry to wash any clothing that may be infested with lice to aid in the extermination process of body and pubic lice.

Accordingly, besides the advantages of the compositions and methods described in this specification, several advantages of one or more aspects include, without limitation:
a) To provide novel methods for treating all forms of human louse infestations.
b) To provide protection from infestation during lice outbreaks.
c) To provide a preventive shampoo than can be used daily in place of regular shampoo, thereby creating no disruption to daily routine.
d) To provide louse prevention that leaves the hair and body pleasant feeling, and not greasy like silicone-based products.
e) To provide louse prophylaxis that doesn't have an overwhelming odor.
f) To provide an easy to use alternative to complicated treatment regimens.
g) To provide a louse control treatment that doesn't require anything outside of the ordinary.
h) To provide a ready-to-use treatment that can be used as much and as often as desired.
i) To provide a non-toxic alternative to conventional toxic pesticides.
j) To provide for novel alternative methods of treatment and prevention of pediculosis.
k) To provide a safe lice treatment.
l) To provide an effective way to both prevent and treat louse infestations.
m) To provide a lice treatment and prophylactic that is generally recognized as safe for its intended purpose.
n) To provide an easy way to control and prevent louse infestations.
o) To provide a treatment that can be used daily to control any newly contracted lice.
p) To provide lice control on human hosts as well as clothing and linens.
q) To provide a method of lice prevention without the use of leave-in repellents.
r) To provide a shampoo and conditioner in one that also controls head lice.
s) To provide a lice control shampoo that's safe for daily use on the entire family, thereby preventing family outbreaks.
t) To provide a means of making a person's hair and body uninhabitable to lice.
u) To provide a means of deterring lice from laying eggs on a person using the composition daily due to maintaining an undesirable egg-laying environment.
v) To provide a moisture-rich composition that nourishes the hair and scalp while it controls louse populations.
w) To provide a treatment and prevention option that is easily removed from the head and body.
x) To provide a treatment and prevention option that can be easily and quickly used on any and all exterior body parts.
y) To provide a treatment and prevention alternative which children can safely apply to themselves during their normal daily routine.
z) To provide an easy way for treating and preventing louse infestations on oneself
aa) To provide uncomplicated methods for effective elimination and prevention of louse infestations.
bb) To provide an FDA compliant homeopathic drug for the control and prophylaxis of pediculosis that is not subject to regulatory action.
cc) To provide a method of obtaining an elegant, stable, foaming composition with an exceptionally high oil content.
dd) To provide an alternate method of adding ingredients while formulating soaps/shampoos.

In broad embodiment, the present invention is a viscous liquid composition to control and prevent louse populations when applied directly to the wetted affected area, worked into a lather, rinsed, then repeated.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A non-toxic stable composition having pediculicidal and prophylactic properties comprising:
a. from about 0.01 to 10 weight percent of at least one ionic salt;
b. from about 1 to about 20 weight percent of at least one natural or synthetic oil;
c. from about 0.1 to about 25 weight percent of at least one conditioning agent;
d. from about 1 to about 10 weight percent of at least one fragrance fixative;
e. from about 1 to about 50 weight percent of at least one surfactant or combination of surfactants;
f. from about 0.1 to about 5 weight percent of at least one plant essence or combination of plant essences;
g. from about 0.1 to about 5 weight percent of an optional chelating agent;
h. from about 0.1 to about 0.5 weight percent of an optional fragrance or combination of fragrances; and
i. water

2. The composition of claim 1 wherein the salt is sodium chloride.

3. The composition of claim 1 wherein the natural or synthetic oil is coconut oil.

4. The composition of claim 1 wherein the conditioning agent is fractionated coconut oil.

5. The composition of claim 1 wherein the fragrance fixative is benzyl alcohol.

6. The composition of claim 1 wherein the surfactant is a combination of cocomidopropyl betaine and 70% active sodium laureth sulfate.

7. The composition of claim 1 wherein the plant essence is a combination of tea tree oil, lavender oil, anise oil, and peppermint oil;

8. The composition of claim 1 wherein the optional chelating agent is disodium EDTA.

9. The composition of claim 1 wherein the optional fragrance is ylang ylang oil.

10. The composition of claim 1 wherein said composition strips away louse exoskeletons, leaving them vulnerable to the environment.

11. The composition of claim 1 wherein said composition controls louse populations on human hair and body parts.

12. The composition of claim 1 wherein said composition creates an environment unfit for louse habitation.

13. The composition of claim 1 wherein said composition controls louse populations on clothing.

14. A method of controlling louse infestations of human hair and body parts comprising:
a. applying the composition of claim 1 to wet hair or body parts;
b. working into a lather then rinsing, whereby the louse exoskeleton is stripped of its protective waxy covering; and
c. repeating said application, lathering, and rinsing process whereby exposed lice are exterminated or permanently damaged.

15. A method of preventing louse infestations of human hair and body parts comprising:
a. applying the composition of claim 1 to wet hair or body parts;
b. working into a lather then rinsing,
c. repeating said application, lathering, and rinsing process whereby the hair and body are left in a state unfit for louse habitation.

16. A method for controlling louse populations on washable clothing comprising adding a small amount of the composition of claim 1 to the laundry and washing the infested clothing whereby the lice are exterminated.
